(19) United States
(12) Patent Application Publication
(10) Pub. No.: US 2011/0098634 A1
(43) Pub. Date: Apr. 28, 2011

(54) TOPICAL ANESTHETIC AND ANTISEPTIC DISPENSING DEVICE

(76) Inventor: Michael Wycoki, Hobe Sound, FL (US)

(21) Appl. No.: 12/981,924

(22) Filed: Dec. 30, 2010

Related U.S. Application Data
(63) Continuation-in-part of application No. 12/388,347, filed on Feb. 18, 2009.

(51) Int. Cl. A61M 5/42; A61B 17/3211

(52) U.S. Cl. 604/22; 604/112

(57) ABSTRACT

Embodiments of the present invention provide a topical anesthetic and antiseptic dispensing device article of manufacture. In an embodiment of the invention, the article of manufacture includes a penetrating instrument including an elongated body with first and second distal end such that at least one of the distal ends includes a penetrating edge. By way of example, the penetrating instrument can be a syringe, an IV catheter needle assembly, a pressurized tube holder configured with a needle for blood draw, or a scalpel, to name a few possibilities. The article of manufacture further includes an attachment connector to couple the container to the housing of the penetrating instrument, where the attachment connector includes a circular connector body and a connector arm, the connector arm affixed to a spray housing of the dispensing device. The container provides at least one chamber. Finally, the article of manufacture includes a nozzle connected to the container and configured to dispense pressurized content of the container responsive to activating a trigger assembly communicating with the nozzle.

### RELATED APPLICATIONS

This application is a continuation-in-part of pending U.S. application Ser. No. 12/388,347 filed Feb. 18, 2009 to Michael Wycoki, entitled TOPICAL ANESTHETIC AND ANTISEPTIC DISPENSING DEVICE, the entire contents of which are incorporated herein by reference.

### BACKGROUND OF THE INVENTION

1. Field of the Invention

The present invention relates to the field of anesthetic and anesthetic dispensing devices and more particularly to aerosol and pressurized type topical anesthetic and anesthetic single dose dispensing devices.

2. Description of the Related Art

Penetrating instruments like scalpels, knives, intravenous (IV) needle assemblies and syringes form an integral part of medical care. One cannot imagine health care without the use of a penetrating instrument such as a needle for use in an injection, or a scalpel for use in a surgical procedure. Indeed, even the most simplistic of diagnostic techniques—the blood draw—requires the use of a needle (ordinarily included as part of a pressurized tube holder configured for blood draw). Even still, excessive pain and adverse reactions associated with the use of a penetrating instrument can cause patient anxiety and unnecessary distress. Consequently, health care providers constantly seek methods and tools to alleviate the pain and reaction caused by the use of a penetrating instrument.

In this regard, it is well known that spraying ethyl chloride on a target zone of a patient for a penetrating instrument can greatly reduce penetrating instrument related pain. Specifically, ethyl chloride is a topical anesthetic. When topically applied to the skin, ethyl chloride creates an instantaneous anesthetic effect on the surface of the site caused by the immediate evaporation of the ethyl chloride. As a result, the cooling effect created by spraying ethyl chloride on a target zone of a patient interrupts the ability of the patient to experience pain resulting from the use of the penetrating instrument on the target zone.

Yet, the anesthetic effect of ethyl chloride generally lasts only upwards of a few seconds. Thus, unless the health care provider is quick to apply the penetrating instrument immediately following the application of ethyl chloride, the ethyl chloride may have to be reapplied. Thus, typically a nurse will clean the target area with an antiseptic, position all equipment and instruments ready for the planned procedure and then apply the ethyl chloride immediately before performing the planned procedure. Accordingly, the described protocol of sterilizing the target zone, preparing the equipment and instruments and applying ethyl chloride can increase the overall time a nurse must spend with each patient. Further, generally an additional person is required in order to complete the anesthetic and antiseptic protocol described herein.

Of note, by having a second nurse present to apply the ethyl chloride in concert with a first nurse performing the expected procedure with the use of a penetrating instrument, the overall time spent on a patient can decrease, thus increasing the capacity to perform more procedures on more patients. However, an additional nurse can be costly. Further, coordinating the placement of the ethyl chloride with the anticipated placement of a needle between two nurses can be problematic in that one nurse may not completely recognize the intention of the other nurse in respect to the placement of the penetrating instrument. Even still, having only a single nurse sterilize the target zone, apply the ethyl chloride to the target zone and then perform the planned procedure can result in the unnecessary waste of time when multiple applications of ethyl chloride is required.

### BRIEF SUMMARY OF THE INVENTION

Embodiments of the present invention provide a topical anesthetic and antiseptic dispensing device article of manufacture. In an embodiment of the invention, the article of manufacture includes a penetrating instrument including a housing with first and second distal end such that at least one of the distal ends includes a penetrating edge. By way of example, the penetrating instrument can be a syringe, an IV catheter needle assembly, a pressurized tube holder configured with a needle for blood draw, or a scalpel, to name a few possibilities. The article of manufacture also includes an attachment connector which couples the container to the housing of the penetrating instrument, the attachment connector includes a circular connector body and a connector arm, where the connector arm is affixed to a spray housing of the dispensing device. The connector body has an inner portion that is tapered and configured to receive a connection segment of the penetrating instrument. The container provides at least one chamber. Finally, the article of manufacture includes a nozzle connected to the container and configured to dispense pressurized content of the container responsive to activating a trigger communicating with the nozzle.

In one aspect of the embodiment of the invention, the chamber contains a pressurized combination of ethyl chloride and alcohol. In another aspect of the embodiment, the chamber contains a pressurized combination of ethyl chloride and povidone iodine (e.g., BETADINE®). In yet another aspect of the embodiment, the container includes two chambers, each chamber containing different pressurized content. In respect to the latter aspect of the embodiment, one chamber contains pressurized ethyl chloride and another chamber contains alcohol. Alternatively, one chamber contains pressurized ethyl chloride and another chamber contains povidone iodine. Further in respect to the latter aspect of the embodiment, the nozzle can be a bi-valve nozzle with two openings, each of the two openings corresponding to a different one of the two chambers.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1A is a perspective view of a topical anesthetic and antiseptic dispensing device article of manufacture coupled to a syringe.

FIG. 1B is a perspective view of a topical anesthetic and antiseptic dispensing device article of manufacture coupled to a scalpel.

FIG. 1C is a perspective view of a topical anesthetic and antiseptic dispensing device article of manufacture coupled to a pressurized tube holder configured for blood draw.

FIG. 1D is a perspective view of a topical anesthetic and antiseptic dispensing device article of manufacture coupled to an IV catheter.

FIG. 1E is a perspective view of a topical anesthetic and antiseptic dispensing device article of manufacture as a stand-alone hand-held unit.

FIG. 2 is a component part perspective view of a topical anesthetic and antiseptic dispensing device article of manufacture.

FIG. 3 is a side view sequence illustration of a topical anesthetic and antiseptic dispensing device article of manufacture in use.

FIG. 4B is a cross-sectional view of the interior construction of a dual chamber container of a topical anesthetic and antiseptic dispensing device article of manufacture of FIG. 4A

FIG. 5B is a cross-sectional view of the interior construction of a single chamber container of a topical anesthetic and antiseptic dispensing device article of manufacture of FIG. 5A.

FIG. 6 illustrates a perspective view of another embodiment of a penetrating instrument (i.e., a syringe) with a topical anesthetic and antiseptic dispensing device article of manufacture.

FIG. 7 illustrates an exploded perspective view of the syringe with a topical anesthetic and antiseptic dispensing device article of manufacture of FIG. 6.

FIG. 8 illustrates a cross-sectional view of the syringe with a topical anesthetic and antiseptic dispensing device article of manufacture of FIG. 6 taken along the A-A plane.

FIG. 9 illustrates a perspective view of another embodiment of a penetrating instrument (i.e., an IV catheter needle assembly) with a topical anesthetic and antiseptic dispensing device article of manufacture.

FIG. 10 illustrates a perspective view of another embodiment of a penetrating instrument (i.e., a pressurized tube holder) with a topical anesthetic and antiseptic dispensing device article of manufacture.

FIG. 11 illustrates a perspective view of another embodiment of a penetrating instrument (i.e., a scalpel) with a topical anesthetic and antiseptic dispensing device article of manufacture.

### DETAILED DESCRIPTION OF THE INVENTION

Embodiments of the present invention provide a topical anesthetic and antiseptic dispensing device article of manufacture. The dispensing device article of manufacture includes a container with one or more chambers. At least one of the chambers can contain a pressurized chemical providing at least an anesthetic effect when released onto a target zone of a patient, and optionally an antiseptic effect when released onto the target zone. For example, the chemicals can include ethyl chloride or a combination of ethyl chloride and alcohol, or ethyl chloride and povidone iodine (e.g., BETADINE®). Optionally, the container can be a modular container accepting one or more canisters of pressurized chemicals such that each canister accounts for a chamber of pressurized chemicals and at least one of the canisters can include a pressurized form of ethyl chloride.

Of note, the container can be adapted for coupling to a penetrating instrument, such as a syringe, scalpel, knife, pressurized tube holder configured for blood draw or an IV catheter needle assembly. In this way, an anesthetic (and optionally an antiseptic) can be released onto the target zone of a patient within close temporal proximity of the use of the coupled penetrating instrument without requiring multiple health care providers to both apply the anesthetic and to use the penetrating instrument.

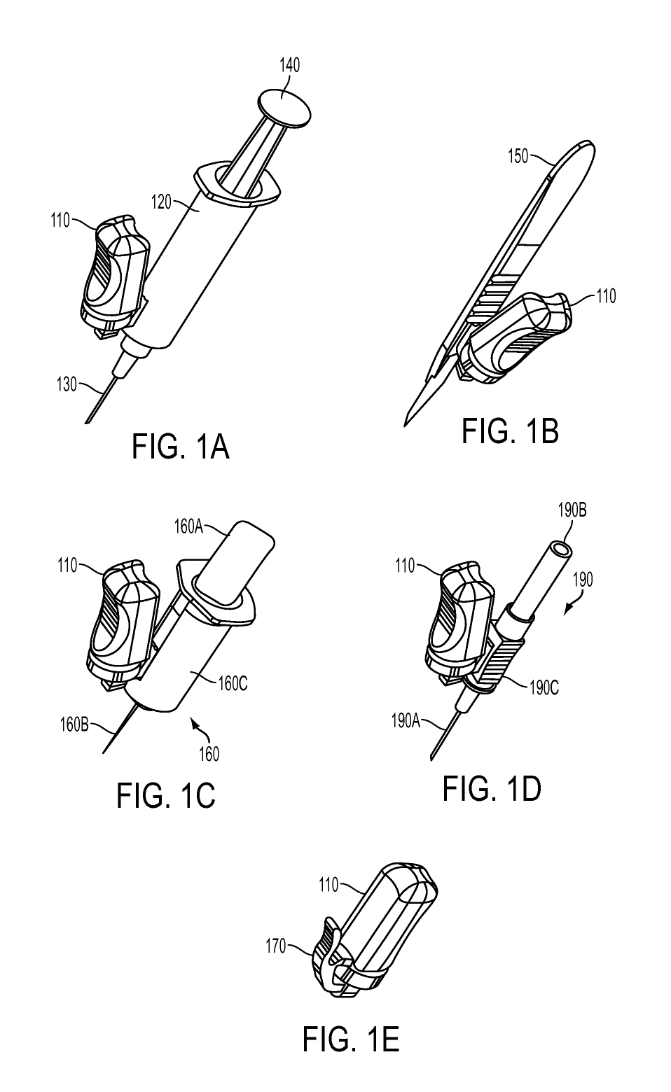

In further illustration, FIGS. 1A through 1D are perspective views of a topical anesthetic and antiseptic dispensing device article of manufacture. As shown in each of FIGS. 1A through 1D, a container 110 can be attached to a penetrating instrument 120, 150, 160, 170 having an elongated body and distal ends opposite to one another with respect to the elongated body. For instance, as shown in FIG. 1A, the penetrating instrument can be a syringe 120 having a pump 140 at one end and a needle 130 at the opposite end. Alternatively, the container 110 can be configured to adapt to a scalpel 150 as shown in FIG. 1B. Another configuration shown in FIG. 1C depicts the container 110 coupled to a pressurized tube holder configured for blood draw 160. The pressurized tube holder configured for blood draw 160 can include a housing 160C with a needle 160B disposed at one end and a pressurized tube 160A at an opposite end partially disposed in the housing 160C for attachment to an opening in the needle so as to facilitate the collection of blood. Yet, another configuration as shown in FIG. 1D can include the container 110 adapted for coupling to an IV catheter needle assembly 190 including a body 190C, a catheter port 190B at one end of the body 190C and a needle 190A at an opposite end of the body 190C.

In all four exemplary aspects of the embodiment illustrated in FIGS. 1A through 1D, the container 110 can include a rotatable joint disposed between the modular canister 110 and the penetrating instrument 120, 150, 160, 170 such that the depression of the container 110 towards the penetrating instrument acts to create an opening between the atmosphere and the pressurized content contained by the container 110 allowing for the pressurized content to be released into the atmosphere and directed onto a target zone of a patient as directed by a nozzle (not shown). Notwithstanding, as shown in FIG. 1E, the container 110 can be a stand-alone hand-held unit having a trigger handle 170 in order to allow the contents of the modular canister 110 to be released out of the modular canister 110 onto a target zone when the trigger handle 170 is rotated toward the container 110.

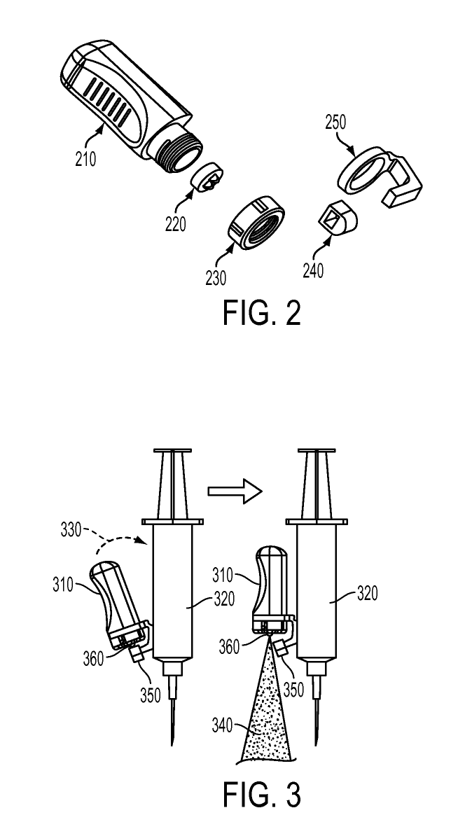

In more particular illustration of an embodiment of the dispensing device article of manufacture, FIG. 2 provides a perspective view of an anesthetic and antiseptic dispensing device article of manufacture. As shown in FIG. 2, the dispensing device article of manufacture can include a container 210 providing at least one chamber containing a pressurized anesthetic. In particular, two separate chambers can be provided, each containing a different chemical, at least one of the chambers containing pressurized ethyl chloride. To facilitate the dispensing of the content of both chambers, a bi-valve nozzle 220 can be attached to the chamber 210 to allow dispensing of the two different chemicals simultaneously. Furthermore, a locking ring 230 with a rubber stop 240 can secure over the bi-valve nozzle 220.

Of note, a trigger 250 can be coupled to the chamber 210 to allow for a pressurized aerosol type triggering mechanism that can evenly dispense the contents of the modular canister 110. Specifically, the trigger 250 can be biased to hold the rubber stop 240 against the bi-valve nozzle 220. When activated, for example rotated, the trigger 250 can permit the rubber stop 240 to release the rubber stop 240 from biased contact against the bi-valve nozzle 220. In consequence, the content of each chamber of the container 210 can be released through the bi-valve nozzle 220 onto a target zone of a patient.

Importantly, the container 210 can be configured for coupling to a penetrating instrument, for example by way of glue or weld or fastener. In illustration, as shown in FIG. 3, the container 310 can be coupled to a penetrating instrument 320 such as a syringe by way of the trigger 350. In this regard, moving the container 310 along a rotating trajectory 330 toward the penetrating instrument 320 can act to dispense the content 340 of the container 310 through nozzle 360. Notably, since the container 310 can include more than one internal chamber, more than one chemical can be sprayed through the nozzle 360 onto a target zone of a patient simultaneously.

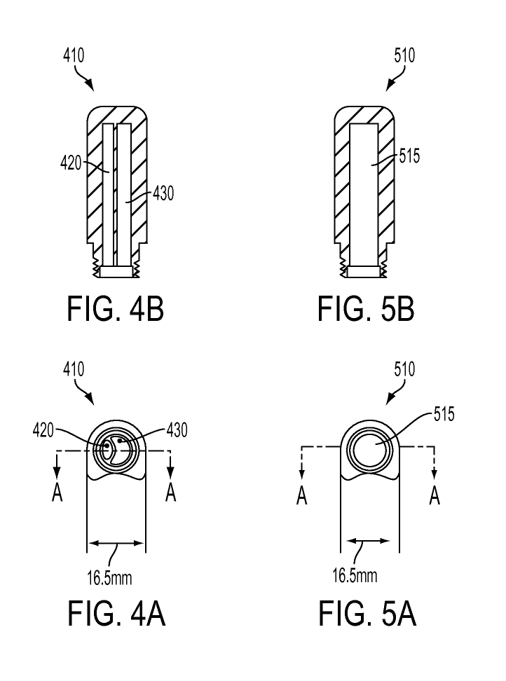

In further illustration, FIG. 4B provides a cross-sectional view of the interior of an anesthetic and antiseptic dispensing device article of manufacture taken along the A-A line of FIG. 4A. The anesthetic and antiseptic dispensing device article of manufacture can include a container 410. The container 410 can include a first chamber 420 and a second chamber 430 separate from the first chamber 420. Preferably, one of the chambers 420 or 430 can contain a pressurized form of a topical anesthetic such as ethyl chloride and the other of the chambers 420 or 430 can contain an antiseptic or other chemical, for instance an analgesic. For example, the container 410 can contain alcohol in one of the chambers 420 or 430 and ethyl chloride in the other of the chambers 420, 430 for dual topical anesthetic and antiseptic use before introducing a penetrating instrument to the target zone of the patient. It will be recognized by one of skill in the art that the chambers 420, 430 can contain equivalent volumes or can contain different volumes. In another embodiment, FIG. 5B provides a cross-sectional view of the interior of a container of an anesthetic and antiseptic dispensing device article of manufacture taken along the A-A line of FIG. 5A. The anesthetic and antiseptic dispensing device article of manufacture can include a container 510, which has a single chamber 515 that contains a combination of ethyl chloride and alcohol, or ethyl chloride and povidone-iodine.

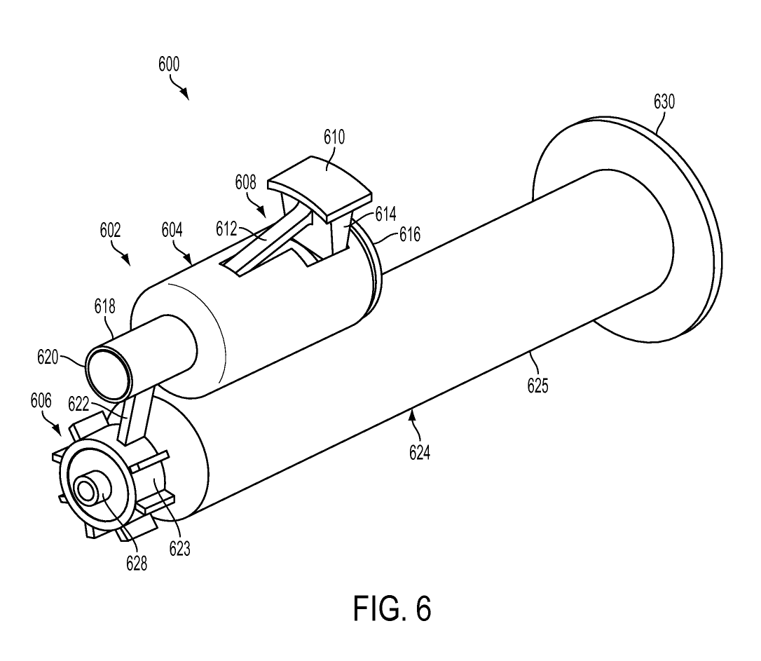

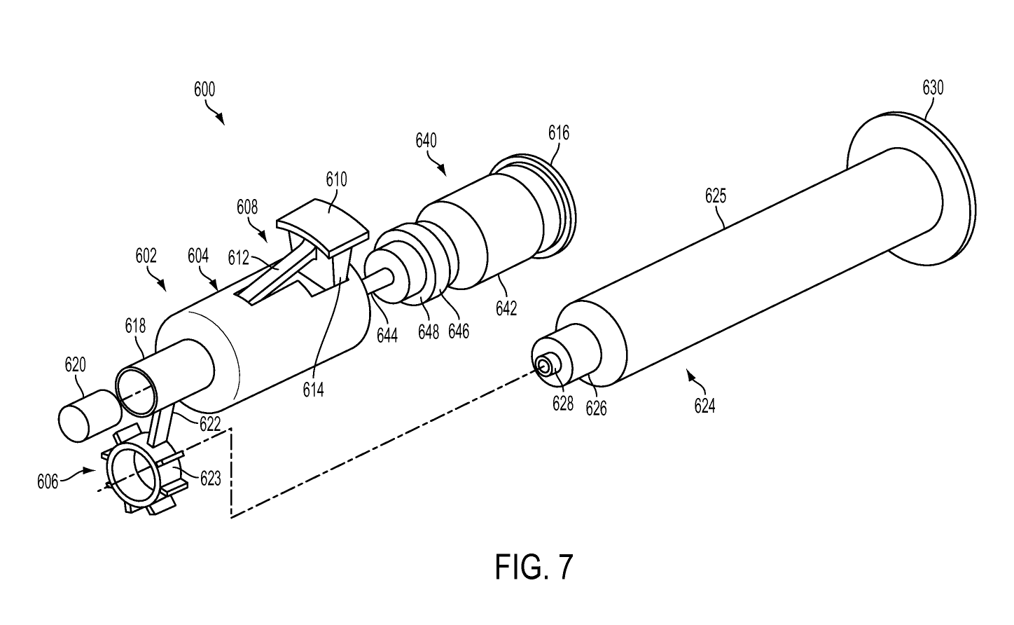

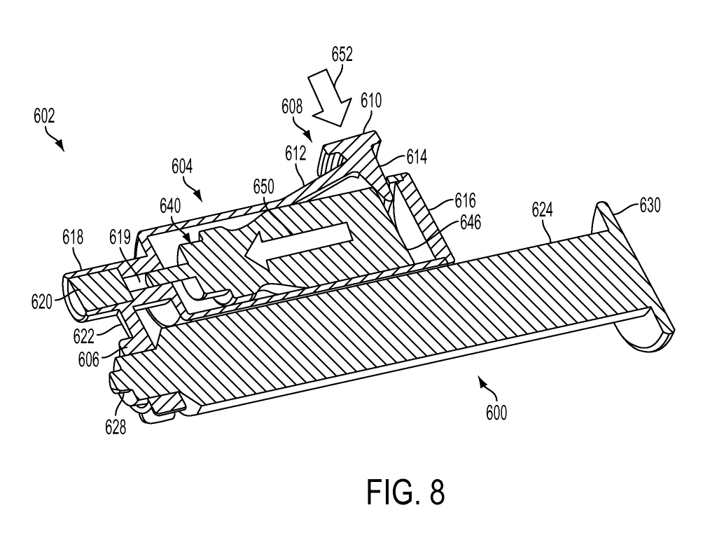

FIG. 6 illustrates a perspective view of another embodiment of a penetrating instrument with a topical anesthetic and antiseptic dispensing device article of manufacture. In this embodiment, penetrating instrument 600 is a syringe attached to dispensing device 602 by an attachment connector 606. Attachment connector 606 includes a connector body 623 and a connector arm 622. In embodiments, the connector body 623 has a circular shape that can include a tapered inside portion designed to receive a tube connection segment 626 (e.g., a male tapered fitting of a Luer lock connector) affixed to a distal end of a syringe housing 624 of syringe 600. Syringe housing 624 can include a syringe flange 630 located at a proximal end opposite the tube connection segment 626, a syringe barrel 625 and an outlet tube 628 extending from tube connection segment 626. In operation, as the tube connection segment 626 is inserted into the connector body 623 it is advanced forward until a friction lock is achieved. Dispensing device 602 further can include a spray housing 604 and a trigger assembly 608. As illustrated in FIGS. 6 and 7, attachment connector 606 is affixed to an outlet tube 618 that is attached to a distal end of spray housing 604. Trigger assembly 608 can include a push button pad 610, a trigger arm 612 and a wedge-shaped member 614. A more detailed discussion of the operation of trigger assembly 608 is provided with respect to FIG. 8. Spray housing 606 defines a compartment for receiving a canister 640. As discussed with respect to FIG. 2, canister 640 provides at least one chamber containing a pressurized anesthetic. In particular, two separate chambers can be provided, each containing a different chemical, at least one of the chambers containing pressurized ethyl chloride. In embodiments, the at least one chamber can contain a combination of ethyl chloride and alcohol, or ethyl chloride and povidone-iodine. In other embodiments, canister 640 can include a single chamber that contains a combination of ethyl chloride and alcohol, or ethyl chloride and povidone-iodine. Canister 640 can include a central housing 642 having a distal outlet portion 644 and a proximal end portion 646 opposite the distal outlet portion 644. Canister 640 can be inserted into the compartment defined by spray housing 606.

FIG. 8 is a cross sectional perspective view of the syringe with a topical anesthetic and antiseptic dispensing device article of manufacture of FIG. 6 taken along the 8-8 plane. FIG. 8 illustrates the operation of trigger assembly 608. In embodiments a push button pad 610 is affixed to wedge-shaped member 614. Wedge-shaped member 614 is affixed to trigger arm 612 and can be rotated or push in a downward direction 652 to cause canister 640 to move forward in the direction 650 of the spray nozzle 620. In this embodiment, there is no force applied to end cap 616, but instead the force is applied to the end portion 646 of canister 640 which causes the canister to advance in a forward direction as illustrated by arrow 650 and spray the contents of the canister 640, for example, onto a treatment area of a patient.

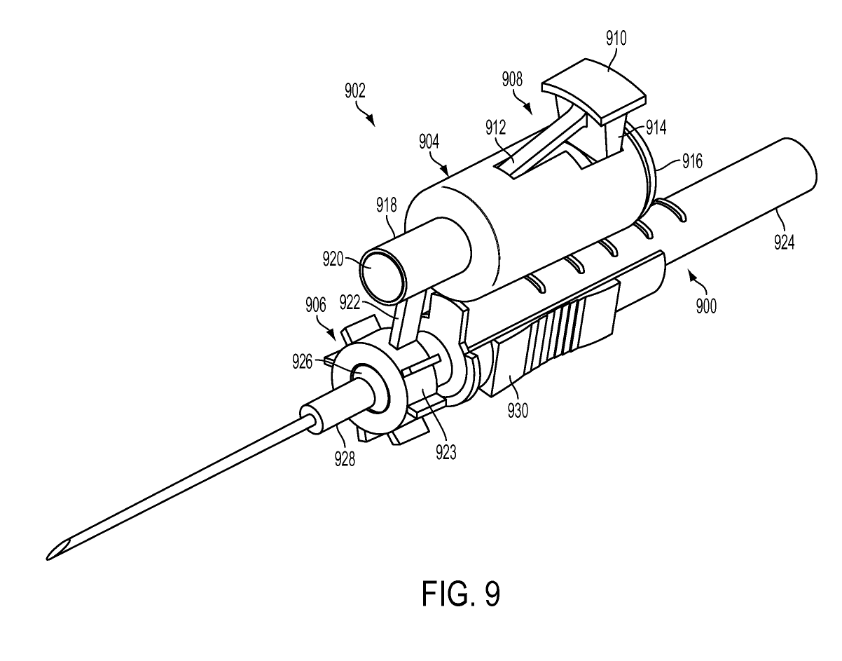

FIG. 9 illustrates a perspective view of another embodiment of a penetrating instrument with a topical anesthetic and antiseptic dispensing device article of manufacture. In this embodiment, penetrating instrument 900 is an IV catheter needle assembly attached to dispensing device 902 by an attachment connector 906. Attachment connector 906 includes a connector body 923 and a connector arm 922. In embodiments, the connector body 923 has a circular shape that can include a tapered inside portion designed to receive a tube connection segment 926 (e.g., a male tapered fitting of a Luer lock connector) affixed to a distal end of an IV catheter housing 924 of syringe 900. IV catheter housing 924 can include a catheter port 925 located at a proximal end opposite the tube connection segment 926 and an outlet tube 928 extending from tube connection segment 926. In operation, as the tube connection segment 926 is inserted into the connector body 923 it is advanced forward until a friction lock is achieved. Dispensing device 902 further can include a spray housing 904 and a trigger assembly 908. Similar to the discussion of FIGS. 6 and 7, attachment connector 906 is affixed to an outlet tube 918 that is attached to a distal end of spray housing 904. Trigger assembly 908 can include a push button pad 910, a trigger arm 912 and a wedge-shaped member 914. Spray housing 906 defines a compartment for receiving a canister 640. As discussed with respect to FIG. 2, canister 640 provides at least one chamber containing a pressurized anesthetic. In embodiments, two separate chambers can be provided, each containing a different chemical, where at least one of the chambers containing pressurized ethyl chloride. In embodiments, the at least one chamber can contain a combination of ethyl chloride and alcohol, or ethyl chloride and povidone-iodine. In other embodiments, canister 640 can include a single chamber that contains a combination of ethyl chloride and alcohol, or ethyl chloride and povidone-iodine. Canister 640 can include a central housing 642 having a distal outlet portion 644 and a proximal end portion 646 opposite the distal outlet portion 644. Canister 640 can be inserted into the compartment defined by spray housing 906.

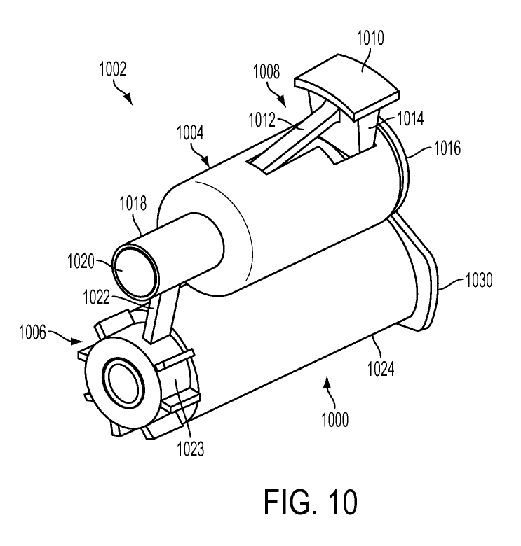

FIG. 10 illustrates a perspective view of another embodiment of a penetrating instrument with a topical anesthetic and antiseptic dispensing device article of manufacture. In this embodiment, penetrating instrument 1000 is a pressurized tube holder attached to dispensing device 1002 by an attachment connector 1006. Attachment connector 1006 includes a connector body 1023 and a connector arm 1022. In embodiments, the connector body 1023 has a circular shape that can include a tapered inside portion designed to receive a tube connection segment 1026 (e.g., a male tapered fitting of a Luer lock connector) affixed to a distal end of a pressurized tube holder housing 1024 of pressurized tube holder 1000. Pressurized tube holder housing 1024 can include a tube port 1025 located at a proximal end opposite the tube connection segment 1026 and an outlet tube 1028 extending from tube connection segment 1026. In operation, as the tube connection segment 1026 is inserted into the connector body 1023 it is advanced forward until a friction lock is achieved. Dispensing device 1002 further can include a spray housing 1004 and a trigger assembly 1008. Similar to the discussion of FIGS. 6 and 7, attachment connector 1006 is affixed to an outlet tube 1018 that is attached to a distal end of spray housing 1004. Trigger assembly 1008 can include a push button pad 1010, a trigger arm 1012 and a wedge-shaped member 1014. Spray housing 1006 defines a compartment for receiving a canister 640. As discussed with respect to FIG. 2, canister 640 provides at least one chamber containing a pressurized anesthetic. In embodiments, two separate chambers can be provided, each containing a different chemical, at least one of the chambers containing pressurized ethyl chloride. In embodiments, the at least one chamber can contain a combination of ethyl chloride and alcohol, or ethyl chloride and povidone-iodine. In other embodiments, canister 640 can include a single chamber that contains a combination of ethyl chloride and alcohol, or ethyl chloride and povidone-iodine. Canister 640 can include a central housing 642 having a distal outlet portion 644 and a proximal end portion 646 opposite the distal outlet portion 644. Canister 640 can be inserted into the compartment defined by spray housing 1006.

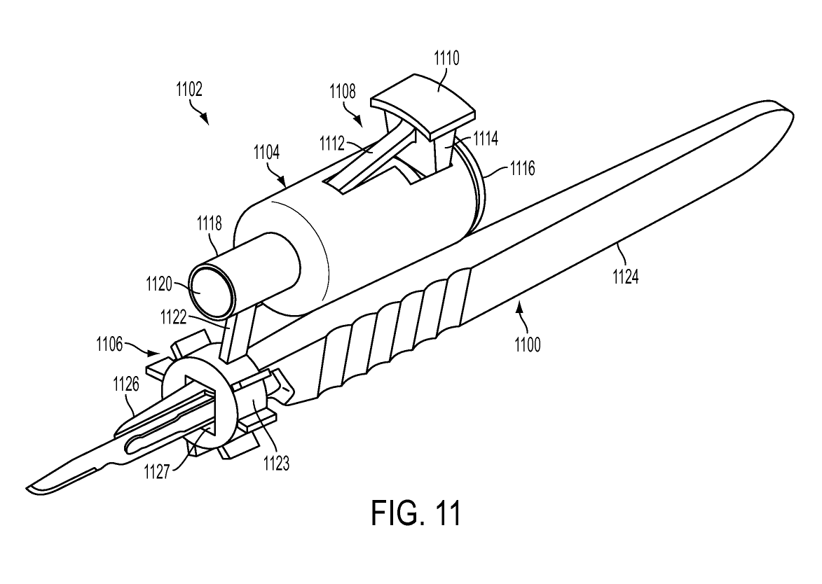

FIG. 11 illustrates a perspective view of another embodiment of a penetrating instrument with a topical anesthetic and antiseptic dispensing device article of manufacture. In this embodiment, penetrating instrument 1100 is a scalpel attached to dispensing device 1102 by an attachment connector 1106. Attachment connector 1106 includes a connector body 1123 and a connector arm 1122. In embodiments, the connector body 1123 has a circular shape that can include a tapered inner portion designed to receive a scalpel connection segment 1126 (e.g., a male tapered fitting of a Luer lock connector) affixed to a distal end of a scalpel housing 1124 of scalpel 1100. In embodiments, the tapered inner portion 1127 can be an insert that has the shape of a square or rectangle and configured to receive a distal end portion of scalpel housing 1124. Scalpel housing 1124 can include a catheter handle 1125 located at a proximal end opposite the connection segment 1126. In operation, as the scalpel connection segment 1126 is inserted into the connector body 1123 it is advanced forward until a friction lock is achieved. Dispensing device 1102 further can include a spray housing 1104 and a trigger assembly 1108. Similar to the discussion of FIGS. 6 and 7, attachment connector 1106 is affixed to an outlet tube 1118 that is attached to a distal end of spray housing 1104. Trigger assembly 1108 can include a push button pad 1110 affixed to a wedge-shaped member 1114, the wedge-shaped member 1114 affixed to rotatable trigger arm 1112. Spray housing 1106 defines a compartment for receiving a canister 640. As discussed with respect to FIG. 2, canister 640 provides at least one chamber containing a pressurized anesthetic. In particular, two separate chambers can be provided, each containing a different chemical, at least one of the chambers containing pressurized ethyl chloride. In embodiments, the at least one chamber can contain a combination of ethyl chloride and alcohol, or ethyl chloride and povidone-iodine. In other embodiments, canister 640 can include a single chamber that contains a combination of ethyl chloride and alcohol, or ethyl chloride and povidone-iodine. Canister 640 can include a central housing 642 having a distal outlet portion 644 and a proximal end portion 646 opposite the distal outlet portion 644. Canister 640 can be inserted into the compartment defined by spray housing 1106.

The invention has been described with respect to certain preferred embodiments, but the invention is not limited only to the particular constructions disclosed and shown in the drawings as examples, and also comprises the subject matter and such reasonable modifications or equivalents as are encompassed within the scope of the appended claims.

I claim:

1. A topical anesthetic and antiseptic dose dispensing device article of manufacture comprising:
a penetrating instrument comprising a housing with first and second distal ends, at least one of the distal ends comprising a penetrating edge;
a container externally coupled to the housing of the penetrating instrument the container providing at least one chamber;
an attachment connector to couple the container to the housing of the penetrating instrument, the attachment connector including a circular connector body and a connector arm, the connector arm connected to a spray housing of the dispensing device; and
a nozzle connected to the container and configured to dispense pressurized content of the at least one chamber responsive to activating a trigger assembly communicating with the nozzle.

2. The article of manufacture of claim 1, wherein the single container provides two chambers, each chamber containing different pressurized content.

3. The article of manufacture of claim 1, wherein the at least one chamber contains a pressurized combination of ethyl chloride and alcohol.

4. The article of manufacture of claim 1, wherein the at least one chamber contains a pressurized combination of ethyl chloride and povidone iodine.

5. The article of manufacture of claim 2, wherein one chamber contains pressurized ethyl chloride and another chamber contains alcohol.

6. The article of manufacture of 2, wherein one chamber contains pressurized ethyl chloride and another chamber contains povidone iodine.

7. The article of manufacture of claim 2, wherein the nozzle comprises a bi-valve nozzle with two openings, each of the two openings corresponding to a different one of the two chambers.

8. The article of manufacture of claim 1, wherein the penetrating instrument is a syringe.

9. The article of manufacture of claim 1, wherein the penetrating instrument is an intravenous (IV) catheter needle assembly.

10. The article of manufacture of claim 1, wherein the penetrating instrument is a pressurized tube holder configured with a needle for blood draw.

11. The article of manufacture of claim 1, wherein the penetrating instrument is a scalpel.

12. The article of manufacture of claim 1, wherein the connector body has an inner portion that is tapered and configured to receive a connection segment of the penetrating instrument.

13. The article of manufacture of claim 1, wherein the trigger assembly comprises a push button pad affixed to a wedge-shaped member, the wedge-shaped member affixed to a rotatable trigger arm and operable to advance the canister.
